(19) United States
(12) Patent Application Publication
(10) Pub. No.: US 2006/0094764 A1
(43) Pub. Date: May 4, 2006

(54) Cyanothiophenes, their preparation and their use in pharmaceutical compositions

(75) Inventors: Ralf Anderskewitz, Laupheim (DE); Gerd Morschhaeuser, Biberach (DE); Ruediger Streicher, Biberach (DE); Thomas Trieselmann, Warthausen (DE); Rainer Walter, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/250,018

(22) Filed: Oct. 13, 2005

(30) Foreign Application Priority Data
Oct. 21, 2004 (DE) 10 2004 051 188

(51) Int. Cl. A61K 31/4436; A61K 31/381; C07D 333/38; C07D 409/02

(52) U.S. Cl. 514/342; 514/447; 549/61; 546/280.4

(57) ABSTRACT

The present invention relates to cyanothiophenes of general formula 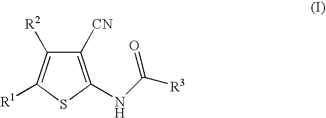 wherein the Rs are defined as in the claims, the tautomers, their stereoisomers, the mixtures thereof and the salts thereof, which have valuable pharmacological properties, particularly a glugacon receptor-antagonistic activity.

### RELATED APPLICATIONS

This application claims priority to German Application No. 10 2004 051 188.8 filed Oct. 21, 2004, which is hereby incorporated by reference in its entirety.

### FIELD OF THE INVENTION

The present invention relates to substituted cyanothiophenes, the preparation thereof, and their use in the preparation of a medicament for the treatment or prevention of diseases involving glucagon receptors.

Diabetes is a complex disease characterised by hyperglycaemia caused by a lack of insulin production or insufficient insulin activity. The metabolic complications of diabetes, hyperglycemia and ketosis, are linked to the relative or absolute increase in the ratio of glucagon to insulin. Consequently, glucagon is a hyperglycemic factor which brings about the rise in the blood sugar.

Therefore, suitable antagonists which block the glucagon receptor are agents for treating diabetes, by inhibiting the production of glucose in the liver and reducing the glucose levels in the patient.

Various publications disclose peptidic and non-peptidic glucagon receptor antagonists (McCormick et al., Curr. Pharm. Des. 7, 1451 (2001), a summary). In particular, the inhibition of glucagon-stimulated glucose production in humans by Bay 27-9955 has been reported (Petersen et al., Diabetologia 44, 2018 (2001)).

The aim of the present invention was to indicate new non-peptidic active substances which are suitable as highly effective glucagon receptor antagonists for the treatment of diabetes.

Cyanothiophenes and their use as glucagon receptor antagonists are already known. Thus, for example, in U.S. Patent Application Publication Nos. 2004/0097552 and 2004/0097557, substituted cyanothiophenes are described which are substituted in the 2 position by an amide, alkylcarbonylamino, arylcarbonylamino, heteroarylcarbonylamino, or heterocyclylamino group.

Moreover, bicyclic heterocycles with a glucagon receptor-antagonistic activity are disclosed in international applications WO 2004/024066 and WO 2004/024065.

Surprisingly it has now been found that 3-cyanothiophenes which carry a halogen atom or a cyano, nitro, or alkoxy group in the 4 position and/or are substituted in the 2 position by a bicycloalkylcarbonylamino group are highly effective glucagon receptor antagonists, which are particularly suitable for preparing pharmaceutical compositions.
The present invention thus relates to the compounds of general formula (I) 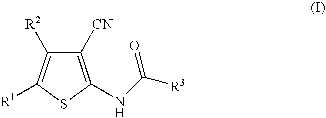
the tautomers, enantiomers, diastereomers, the mixtures thereof, and the salts thereof, particularly the physiologically acceptable salts thereof with inorganic or organic acids or bases which have valuable pharmacological properties, particularly an inhibitory effect on glucagon receptors, their use for the treatment and prevention of diseases, particularly diabetes, and their production.
In the above formula (I):
R¹ denotes a C₁₋₄-alkyl group which may be terminally substituted by a fluorine, chlorine, bromine or iodine atom or by an amino group,
while the hydrogen atoms of the amino group may be substituted independently of one another by a C₁₋₃-alkyl group or a phenyl or pyridyl group optionally substituted by a C₁₋₃-alkyl group,
   or a phenyl or pyridyl group which may be substituted in each case by one to three C₁₋₃-alkyl groups;
   R₂ denotes a hydrogen, fluorine, chlorine, bromine or iodine atom or a cyano, nitro, C₁₋₃-alkyl, trifluoromethyl or C₁₋₄-alkyloxy group; and
   R³ denotes a bicyclo[2.2.1]hept-2-enyl group or
   a cyclohexyl group wherein the carbon atom in position 2 may be bridged to the carbon atom in position 5 by a —CH₂—, —CH₂—CH₂—, or —CH═CH— group.
   Preferred compounds of general formula (I) are those wherein:
   R¹ denotes a C₁₋₄-alkyl group which may be terminally substituted by a fluorine, chlorine, bromine, or iodine atom or by an amino group,
while the hydrogen atoms of the amino group may be substituted independently of one another by a C₁₋₃-alkyl group or a phenyl or pyridyl group optionally substituted by a C₁₋₃-alkyl group,
   or a phenyl or pyridyl group which may be substituted in each case by one to three C₁₋₃-alkyl groups;
   R² denotes a fluorine, chlorine, bromine, or iodine atom or a cyano, nitro, C₁₋₃-alkyl, trifluoromethyl, or C₁₋₄-alkyloxy group; and
   R³ denotes a bicyclo[2.2.1]hept-2-enyl group or
   a cyclohexyl group wherein the carbon atom in position 2 may be bridged to the carbon atom in position 5 by a —CH₂—, —CH₂—CH₂—, or —CH═CH— group,
   the tautomers, enantiomers, diastereomers, the mixtures thereof, and the salts thereof.
   Particularly preferred are those compounds of general formula (I), wherein:
   R¹ denotes a C₁₋₄-alkyl group which may be terminally substituted by a fluorine, chlorine, bromine, or iodine atom or by an amino group,
while the hydrogen atoms of the amino group may be substituted independently of one another by a C₁₋₃-alkyl group or a phenyl or pyridyl group optionally substituted by a C₁₋₃-alkyl group,
   or a phenyl or pyridyl group which may be substituted in each case by one to three C₁₋₃-alkyl groups,
   R² denotes a fluorine, chlorine, bromine, or iodine atom or a cyano, nitro or C₁₋₄-alkyloxy group; and
   R³ denotes a bicyclo[2.2.1]hept-2-enyl group or
   a cyclohexyl group wherein the carbon atom in position 2 may be bridged to the carbon atom in position 5 by a —CH₂—, —CH₂—CH₂—, or —CH═CH— group,
   the tautomers, enantiomers, diastereomers, the mixtures thereof, and the salts thereof.
   A second preferred subgroup comprises those compounds of general formula (I) wherein:
   R¹ denotes a C₁₋₄-alkyl group which may be terminally substituted by a fluorine, chlorine, bromine, or iodine atom or by an amino group,
while the hydrogen atoms of the amino group may be substituted independently of one another by a C₁₋₃-alkyl group or by a phenyl or pyridyl group optionally substituted by a C₁₋₃-alkyl group,
   or a phenyl or pyridyl group which may be substituted in each case by one to three C₁₋₃-alkyl groups,
   R² denotes a hydrogen, fluorine, chlorine, bromine, or iodine atom or a cyano, nitro, C₁₋₃-alkyl, trifluoromethyl, or C₁₋₄-alkyloxy group; and
   R³ denotes a cyclohexyl group wherein the carbon atom in position 2 may be bridged to the carbon atom in position 5 by a —CH₂ or —CH₂—CH₂ group,
   the tautomers, enantiomers, diastereomers, the mixtures thereof, and the salts thereof,
   but particularly those compounds of general formula (I) wherein:
   R¹ denotes a C₁₋₄-alkyl group which may be terminally substituted by a fluorine, chlorine, bromine, or iodine atom or by an amino group,
while the hydrogen atoms of the amino group may be substituted independently of one another by a C₁₋₃-alkyl group or by a phenyl or pyridyl group optionally substituted by a C₁₋₃-alkyl group,
   or a phenyl or pyridyl group which may be substituted in each case by one or two C₁₋₃-alkyl groups,
   R² denotes a fluorine, chlorine, bromine, or iodine atom or a cyano, nitro, C₁₋₃-alkyl, trifluoromethyl or C₁₋₄-alkyloxy group; and
   R³ denotes a cyclohexyl group wherein the carbon atom in position 2 may be bridged to the carbon atom in position 5 by a —CH₂ or —CH₂—CH₂ group,
   the tautomers, enantiomers, diastereomers, the mixtures thereof, and the salts thereof.
   Most particularly preferred are those compounds of general formula (I), wherein
   R¹ denotes a C₂₋₄-alkyl group which is terminally substituted by an N-phenyl-N-methylamino group,
   or a phenyl group which may be substituted by one or two methyl groups;
   R² denotes a chlorine or bromine atom or a cyano or nitro group; and
   R³ denotes a cyclohexyl group wherein the carbon atom in position 2 may be bridged to the carbon atom in position 5 by a —CH₂ or —CH₂—CH₂ group,
   the tautomers, enantiomers, diastereomers, the mixtures thereof, and the salts thereof.
   Particular mention should be made of the following compounds of general formula (I):
(a) 2-[(1S,2R,4R)-bicyclo[2.2.1]hept-2-ylcarbonylamino]-4-chloro-3-cyano-5-(2,5-dimethylphenyl)thiophene,
(b) 2-[(2R)-bicyclo[2.2.2]oct-2-ylcarbonylamino]-4-chloro-3-cyano-5-(2,5-dimethylphenyl)thiophene,
(c) 4-chloro-3-cyano-2-(cyclohexylcarbonylamino)-5-phenylthiophene,
(d) 4-bromo-3-cyano-2-(cyclohexylcarbonylamino)-5-phenylthiophene,
(e) 3,4-dicyano-2-(cyclohexylcarbonylamino)-5-phenylthiophene,
(f) 3-cyano-2-(cyclohexylcarbonylamino)-4-nitro-5-phenylthiophene,
(g) 2-[(1S,2R,4R)-bicyclo[2.2.1]hept-2-ylcarbonylamino]-4-bromo-3-cyano-5-(2,5-dimethylphenyl)thiophene,
(h) 2-[(1S,2R,4R)-bicyclo[2.2.1]hept-2-ylcarbonylamino]4-bromo-3-cyano-5-{3-[methyl-(4-methylphenyl)amino]propyl}thiophene; and
(i) 2-[(1S,2R,4R)-bicyclo[2.2.1]hept-2-ylcarbonylamino]-3,4-dicyano-5-{3-[methyl-(4-methylphenyl)amino]propyl}thiophene,
   and the salts thereof.
   According to the invention the compounds of general formula (I) are obtained by methods known per se, for example, by the following methods:
   a) reacting a compound of general formula (II) 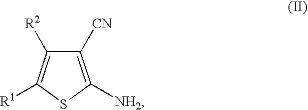
   wherein R¹ and R² are as hereinbefore defined, with an acid chloride R³—COCl of general formula (III), wherein R³ is as hereinbefore defined. The reaction is expediently carried out in a solvent such as methylene chloride, chloroform, carbon tetrachloride, ether, tetrahydrofuran, dioxane, benzene, toluene, acetonitrile, or sulfolane, optionally in the presence of an inorganic or organic base such as, e.g., pyridine or 4-dimethylaminopyridine at temperatures between −20° C. and 200° C., but preferably at temperatures between −10° C. and 160° C.
   b) In order to prepare a compound of formula (I) wherein R² denotes a fluorine, chlorine, bromine, or iodine atom or a nitro group: chlorinating, brominating, iodinating, or nitrating a compound of general formula (IV) 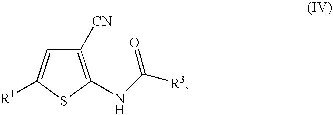
   wherein R¹ and R³ are as hereinbefore defined.
   The chlorination may be carried out, for example, with N-chlorosuccinimide in solvents such as glacial acetic acid, carbon tetrachloride, or dichloromethane at temperatures between 25° C. and 100° C. Alternatively, chlorine, particularly combined with Lewis acids such as, e.g., aluminum chloride, may be used as a reagent.
   The bromination may conveniently be carried out with N-bromosuccinimide in solvents such as glacial acetic acid, carbon tetrachloride, or dichloromethane at temperatures between 25° C. and 100° C., preferably at 60° C.-75° C. Alternatively bromine, particularly combined with Lewis acids such as, e.g., aluminum chloride, may be used as a reagent.
   The iodination may conveniently be carried out with N-iodosuccinimide in solvents such as glacial acetic acid, carbon tetrachloride or dichloromethane at temperatures between 25° C. and 100° C. Alternatively iodine, particularly combined with Lewis acids such as, e.g., aluminum chloride, may be used as a reagent.
   The nitration is conveniently carried out with concentrated nitric acid or nitrating acid in solvents such as, for example, acetic acid or acetic anhydride at temperatures between −5° C. and 40° C., preferably at ambient or room temperature. Alternatively, nitronium tetrafluoroborate in dichloromethane may also be used.
   c) In order to prepare a compound of formula (I), wherein R² denotes a cyano group: reacting a compound of general formula (V) 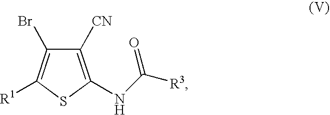
   wherein R¹ and R³ are as hereinbefore defined, with cyanide.
   The reaction is carried out for example with copper (I) cyanide in solvents such as N,N-dimethylformamide, dimethylacetamide, dioxane, benzene, toluene, or acetonitrile with heating, preferably under reflux.
   d) In order to prepare a compound of formula (I) wherein R² denotes a fluorine atom: reacting a compound of general formula (V) 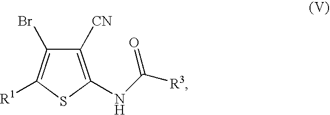
   wherein R¹ and R³ are as hereinbefore defined and wherein no other fluorine, chlorine, bromine, iodine atoms, or nitro groups are present, with n-butyllithium and subsequent reaction with N-fluorodibenzenesulfonimide.
   The reaction with n-butyllithium is carried out in solvents such as diethyl ether or tetrahydrofuran at temperatures below −78° C., preferably in tetrahydrofuran. The subsequent reaction with N-fluorodibenzenesulfonimide is also carried out in solvents such as diethyl ether or tetrahydrofuran at temperatures below −78° C., preferably in tetrahydrofuran.
   e) In order to prepare a compound of formula (I) wherein R¹ denotes a C₁₋₄-alkyl group which may be terminally substituted by an amino group, while the hydrogen atoms of the amino group may be substituted independently of one another by a C₁₋₃-alkyl group or a phenyl or pyridyl group optionally substituted by a C₁₋₃-alkyl group: reacting a compound of general formula (VI) 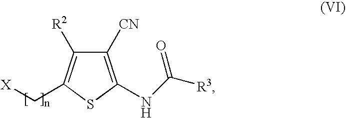
   wherein R² and R³ are as hereinbefore defined and X denotes a leaving group such as, for example, a chlorine, bromine, or iodine atom or a tosyl or triflate group, with a corresponding secondary amine and then optionally converting the substituents on the amino group thus introduced.
   The reaction is conveniently carried out without the addition of any other solvents (apart from the secondary amine) with heating, e.g., by microwave irradiation. Then any protective groups used during the reaction may be cleaved and/or the compounds of general formula (I) thus obtained may be resolved into their enantiomers and/or diastereomers and/or the compounds of formula (I) obtained may be converted into the salts thereof, particularly for pharmaceutical use into the physiologically acceptable salts thereof with inorganic or organic acids.
   Moreover, the compounds of general formula (I) obtained may be resolved into their enantiomers and/or diastereomers, as mentioned hereinbefore. Thus, for example, cis/trans mixtures may be resolved into their cis and trans isomers, and compounds with at least one optically active carbon atom may be separated into their enantiomers.
   Thus, for example, the cis/trans mixtures obtained may be resolved by chromatography into the cis and trans isomers thereof, the compounds of general formula (I) obtained which occur as racemates may be separated by methods known per se (cf. N. L. Allinger and E. L. Eliel in “Topics in Stereochemistry”, Vol. 6, Wiley Interscience, 1971) into their optical antipodes and compounds of general formula (I) with at least 2 asymmetric carbon atoms may be resolved into their diastereomers on the basis of their physical-chemical differences using methods known per se, e.g., by chromatography and/or fractional crystallization, and, if these compounds are obtained in racemic form, they may subsequently be resolved into the enantiomers as mentioned above.
   The enantiomers are preferably separated by column separation on chiral phases or by recrystallization from an optically active solvent or by reacting with an optically active substance which forms salts or derivatives such as, e.g., esters or amides with the racemic compound, particularly acids and the activated derivatives or alcohols thereof, and separating the diastereomeric mixture of salts or derivatives thus obtained, e.g., on the basis of their differences in solubility, whilst the free antipodes may be released from the pure diastereomeric salts or derivatives by the action of suitable agents. Optically active acids in common use are, e.g., the D- and L-forms of tartaric acid or dibenzoyltartaric acid, di-p-toluoyltartaric acid, malic acid, mandelic acid, camphorsulfonic acid, glutamic acid, aspartic acid, or quinic acid. An optically active alcohol may be, for example, (+) or (−)-menthol and an optically active acyl group in amides, for example, may be a (+)-or (−)-menthyloxycarbonyl.
   Furthermore, the compounds of formula (I) may be converted into the salts thereof, particularly for pharmaceutical use into the physiologically acceptable salts with inorganic or organic acids. Acids which may be used for this purpose include, for example, hydrochloric acid, hydrobromic acid, sulfuric acid, methanesulfonic acid, phosphoric acid, fumaric acid, succinic acid, lactic acid, citric acid, tartaric acid, or maleic acid.
   Moreover, if the new compounds of formula (I) thus obtained contain a carboxy group, they may subsequently, if desired, be converted into the salts thereof with inorganic or organic bases, particularly for pharmaceutical use into the physiologically acceptable salts thereof. Suitable bases for this purpose include, for example, sodium hydroxide, potassium hydroxide, cyclohexylamine, ethanolamine, diethanolamine, and triethanolamine.
   The compounds of general formulae (II) to (VI) used as starting materials are either known from the literature or may be obtained by methods known from the literature (cf. Examples I to VIII).
   As already mentioned hereinbefore, the compounds of general formula (I) according to the invention and the physiologically acceptable salts thereof have valuable pharmacological properties, particularly an inhibitory effect on glucagon receptors.
   The biological properties of the new compounds were investigated as follows:
   Glucagon Binding Assay
   The binding of the compounds of formula (I) according to the invention to the glucagon receptor was determined in a displacement binding assay which is based on the displacement of radiolabelled glucagon from a membrane fraction containing the recombinant human glucagon receptor. The cDNA coding for the human glucagon receptor was cloned into the expression vector pcDNA3.1 (Invitrogene). BHK-21 cells (Baby Hamster Kidney C-13 cells, ATCC) were transfected with this construct and a stable cell clone was selected by treatment with G-418 (Gibco) and isolated. A membrane fraction containing the recombinant human glucagon receptor was prepared from this clone by the following steps: Cells growing to confluence were detached using ice-cooled PBS buffer (Gibco) with 0.05% EDTA and suspended. After centrifugation, the pellet was suspended in a buffer (10 mM Tris/HCl, pH 7.2; 0.01 mM PMSF (phenylmethylsulfonylfluoride)) and incubated for 90 minutes at 4° C. After the lysate had been treated with a homogenizer (Dounce), cell nuclei and other cell constituents were separated off by centrifuging at 500 g for 10 minutes. The supernatant was then centrifuged at 100,000 g for 35 minutes to pellet the membranes. The precipitated membranes were suspended in incubation buffer (50 mM Tris/HCl, pH 7.2; 100 mM NaCl; 5 mM MgCl₂; 1 mM EDTA; 0.2% BSA (bovine serum albumin)), aliquoted and stored at −80° C.
   The displacement of glucagon was measured by incubating 20 μg of the membrane fraction, 50.00 cpm of ¹²⁵1-glucagon (Amersham Pharmacia) and a concentration of the test substance for 60 minutes at 20° C. in a volume of 100 μL in incubation buffer in a microtiter plate (Optiplate, Packard Instruments). The bound radioligand was separated from the free ligand by filtration and washing using GC/B filters (Packard) on a Multiscreen vacuum filtration system (Millipore). The measurement was performed using a Topcount scintillation counter (Packard). The binding in the presence of 1 μM of unlabelled glucagon (Wherl GmbH) was defined as non-specific. The data was analyzed so as to determine the percentage of bound activity in the presence of a test substance. The results were calculated as IC₅₀ values. The compounds listed in Examples 1 to 25 give IC₅₀ values of less than 10 μM.
   The glucagon receptor antagonists according to the invention may be administered by oral, transdermal, inhalative, or parenteral route. The compounds according to the invention are present as active ingredients in conventional formulations, for example, in compositions consisting essentially of an inert pharmaceutical carrier and an effective dose of the active substance, such as, for example, tablets, coated tablets, capsules, lozenges, powders, solutions, suspensions, emulsions, syrups, suppositories, transdermal systems, etc. An effective dose of the compounds according to the invention is between 1 and 100, preferably between 1 and 50, most preferably between 5-30 mg/dose for oral administration, and between 0.001 and 50, preferably between 0.1 and 10 mg/dose for intravenous or intramuscular administration. For inhalation, according to the invention, solutions containing 0.01 to 1.0, preferably 0.1 to 0.5% active substance are suitable. For administration by inhalation the use of powders is preferred. It is also possible to use the compounds according to the invention as a solution for infusion, preferably in a physiological saline or nutrient saline solution.
   The compounds according to the invention may be used on their own or in conjunction with other active substances according to the invention, optionally also in conjunction with other pharmacologically active substances selected from among: acarbose, beraprost, bexarotene, captopril, denileukin, diftitox, etanercept, farglitazar, fidarestat, glibenclamide, glibornuride, gliclazide, glimepiride, glipizide, glucagon, ilomastat, imidapril, insulin, lanreotide, linogliride, lisinopril, metformin, mexiletine, miglitol, minalrestat, mitiglinide, moxonidine, nafagrel, nateglinide, octreotide, orlistat, oxcarbazepine, pegvisomant, pioglitazone, ponalrestat, pramlintide, ramipril, repaglinide, rosiglitazone, sirolimus, sorbinil, tolrestat, troglitazone, voglibose, zenarestat, and zopolrestat.
   Suitable preparations include, for example, tablets, capsules, suppositories, solutions, elixirs, emulsions, or dispersible powders. Suitable tablets may be obtained, for example, by mixing the active substance(s) with known excipients, for example, inert diluents such as calcium carbonate, calcium phosphate, or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.
   Coated tablets may be prepared accordingly by coating cores produced analogously to the tablets with substances normally used for tablet coatings, for example, collidone or shellac, gum arabic, talc, titanium dioxide, or sugar. To achieve delayed release or prevent incompatibilities the core may also consist of a number of layers. Similarly the tablet coating may consist of a number or layers to achieve delayed release, possibly using the excipients mentioned above for the tablets.
   Syrups containing the active substances or combinations thereof according to the invention may additionally contain a sweetener such as saccharine, cyclamate, glycerol, or sugar and a flavor enhancer, e.g., a flavoring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.
   Solutions for injection are prepared in the usual way, e.g., with the addition of preservatives such as p-hydroxybenzoates, or stabilizers such as alkali metal salts of ethylenediamine tetraacetic acid, and transferred into injection vials or ampoules.
   Capsules containing one or more active substances or combinations of active substances may for example be prepared by mixing the active substances with inert carriers such as lactose or sorbitol and packing them into gelatine capsules.
   Suitable suppositories may be made, for example, by mixing with carriers provided for this purpose, such as neutral fats or polyethyleneglycol or the derivatives thereof.
   A therapeutically effective daily dose is between 1 and 800 mg, preferably 10 to 300 mg per adult.
   The Examples which follow illustrate the present invention without restricting its scope (abbreviations used: DMF is N,N-dimethylformamide).
   Preparation of the starting compounds:

### EXAMPLE I

### 2-amino-3-cyano-4-trifluoromethyl-5-(2.5-dimethylphenyl)thiophene

### a. 4,4,4-trifluoro-2-(2,5-dimethylphenyl)-3-oxobutyronitrile

26.1 g (145 mmol) of 2,5-dimethylphenylacetonitrile is dissolved in ethanol and combined with 20.2 g (112 mmol) of potassium tert-butoxide. After 30 minutes stirring, 21.4 g (142 mmol) of ethyl trifluoroacetate is added dropwise. Then the mixture is refluxed for 4 hours. The reaction solution is freed from the solvent in vacuo. The residue is combined with water and washed with ether. The aqueous phase is acidified with concentrated hydrochloric acid and extracted with ether. The organic phase is dried over magnesium sulfate and evaporated down. Yield: 34 g (78% of theory); C₁₂H₁₀F₃NO (241.21); mass spectrum: (M−H)⁻=240.

### b. 1,1,1-trifluoro-3-(2,5-dimethylphenyl)-propan-2-one

34 g (241 mmol) of 4,4,4-trifluoro-2-(2,5-dimethylphenyl)-3-oxobutyronitrile is dissolved in 80 mL of glacial acetic acid and combined with 40 mL of 60% sulfuric acid. Then the mixture is refluxed for 30 hours. After the reaction solution has cooled, the glacial acetic acid is distilled off, and the residue is combined with water and extracted with ether. The organic phase is washed with water, 10% sodium carbonate solution and water. After drying on magnesium sulfate, the organic phase is evaporated to dryness. The residue is distilled in vacuo. Yield: 21.3 g (70% of theory); C₁₁H₁₁F₃O (216.20).

### c. 2-amino-3-cyano-4-trifluoro-5-(2,5-dimethylphenyl)thiophene

21.3 g (99 mmol) of 1,1,1-trifluoro-3-(2,5-dimethylphenyl)-propan-2-one is dissolved in 70 mL of ethanol and combined with 6.54 g (99 mmol) of malonic acid dinitrile, 3.17 g (99 mmol) of sulfur, and 11 mL (100 mmol) of N-methylmorpholine. Then the mixture is heated to 120° C. in the microwave (CEM Discovery) for 30 minutes at 200 Watt. Then the solvent is distilled off and the residue is chromatographed on silica gel (toluene/ethyl acetate=9:1). The product is crystallized from petroleum ether/ethyl acetate. Yield: 8 g (27% of theory); C₁₄H₁₁F₃N₂S (296.31); mass spectrum: (M+H)⁺=297.

### EXAMPLE II

### 2-amino-3-cyano-5-(2,5-dimethylphenyl)thiophene

Prepared analogously to Example I by reaction of (2,5-dimethylphenyl)acetaldehyde, malonic acid dinitrile, sulfur, and triethylamine in DMF. C₁₃H₁₂N₂S (228.31); mass spectrum: (M+H)⁺=229.

### EXAMPLE III

### 2-amino-3-cyano-4-trifluoro-5-phenylthiophene

Prepared analogously to Example I by reaction of 1,1,1-trifluoro-3-phenylpropan-2-one, malonic acid dinitrile, sulfur, and N-methylmorpholine in glycol. C₁₂H₇F₃N₂S (268.26); melting point: 172° C.; mass spectrum: (M-H)-=267.

### EXAMPLE IV

### 2-amino-4-bromo-3-cyano-5-(2,5-dimethylphenyl)thiophene

### a. 4,5-dibromo-3-cyano-2-nitrothiophene

4.00 g (10.9 mmol) of 3,4,5-tribromo-2-nitrothiophene and 1.38 g (15.4 mmol) of copper (I) cyanide are dissolved in 10 mL of DMF and stirred for 43 hours at 90° C. Then the solvent is eliminated in vacuo and the residue is combined with 100 mL of water and 100 mL of ethyl acetate. The phases are separated and the aqueous phase is extracted with ethyl acetate. The combined organic phases are washed with water and saturated aqueous sodium chloride solution, dried over sodium sulfate, and concentrated using the rotary evaporator. The residue is chromatographed on silica gel (petroleum ether/ethyl acetate=4:1). Yield: 370 mg (11% of theory); C₅Br₂N₂O₂S (311.94); R_{f}=0.30 (silica gel; petroleum ether/ethyl acetate=9:1).

### b. 4-bromo-3-cyano-5-(2.5-dimethylphenyl)-2-nitrothiophene

50 mg (0.33 mmol) of 2,5-dimethylphenylboric acid and 20 mg (0.027 mmol) of [1,1′-bis-(diphenylphosphino)ferrocene]palladium (II) chloride are added to a solution of 100 mg (0.32 mmol) of 4,5-dibromo-3-cyano-2-nitrothiophene in 2 mL of dioxane. The reaction mixture is stirred for 30 hours at ambient temperature and then combined with 50 mL of water and 50 mL of ethyl acetate. The phases are separated and the aqueous phase is extracted with ethyl acetate. The combined organic phases are dried over sodium sulfate and concentrated using the rotary evaporator. Yield: 55 mg (51% of theory); C₁₃H₉BrN₂O₂S (337.19); R_{f}=0.64 (silica gel; petroleum ether/ethyl acetate=4:1)

### c. 2-amino-4-bromo-3-cyano-5-(2,5-dimethylphenyl)thiophene

1.6 mL (1.2 mmol) of titanium (III) chloride (10% in 20-30% hydrochloric acid) is added at ambient temperature to a solution of 50 mg (0.15 mmol) of 4-bromo-3-cyano-5-(2,5-dimethylphenyl)-2-nitrothiophene in 10 mL of acetone. The reaction mixture is stirred for 1 hour at ambient temperature and then made basic with 10% sodium hydroxide solution. The aqueous phase is extracted with ethyl acetate, and the combined organic phases are dried over sodium sulfate and concentrated using the rotary evaporator. Yield: 27 mg (59% of theory); C₁₃H₁₁BrN₂S (307.21); mass spectrum: (M+H)⁺=309, 307; R_{f}=0.38 (silica gel; petroleum ether/ethyl acetate=4:1)

### EXAMPLE V

### 2-amino-5-(3-chloropropyl)-3-cyanothiophene

Under a nitrogen atmosphere, 5.0 g (24.9 mmol) of 5-chloropentanal is dissolved in 1.64 g (24.9 mmol) of malonic acid dinitrile and combined with 7.43 g (72.8 mmol) of aluminum oxide (basic, activity I) and stirred for 20 minutes at ambient temperature. Then dichloromethane is added and the solid is separated off by suction filtering. The filtrate is dried over magnesium sulfate and freed from the solvent. The crude product thus obtained is dissolved in 5.5 mL of ethanol and combined with 750 mg (23.4 mmol) of sulfur. At ambient temperature, 1.44 mL (13.9 mmol) of diethylamine are added dropwise. Then the reaction solution is stirred for 16 hours at 35° C. Then the solvent is eliminated in vacuo. The residue is chromatographed on silica gel (petroleum ether/ethyl acetate=80:20→63:37). Yield: 781 mg (18% of theory); C₈H₉ClN₂S (200.70); R_{f} value: 0.4 (silica gel; petroleum ether/ethyl acetate=2:1)

### EXAMPLE VI

### 2-amino-5-(3-chloropropyl)-3-cyano-4-methylthiophene

Prepared analogously to Example I from 6-chlorohexan-2-one, malonic acid dinitrile, sulfur, and diethylamine in ethanol. Yield: 26% of theory; CgH₁₁ClN₂S (214.72); mass spectrum: (M+H)⁺=217, 215; R_{f} value: 0.7 (silica gel; petroleum ether/ethyl acetate=1:1).

### EXAMPLE VII

### 2-amino-3-cyano-5-phenylthiophene

Prepared analogously to Example I from phenylacetaldehyde, malonic acid dinitrile, sulfur and N-methylmorpholine in ethanol. Melting point: 177° C.-178° C.; R_{f} value: 0.34 (silica gel; toluene/ethyl acetate=4:1).

### EXAMPLE VIII

### 2-amino-3-cyano-4-methoxy-5-propylthiophene

6.00 g (36.5 mmol) of 2-(1-methoxypentylidene)malonic acid dinitrile is dissolved in 7.5 mL of methanol and combined with 1.20 g (37.4 mmol) of sulfur and 4.02 mL (36.5 mmol) of N-methylmorpholine. The reaction solution is heated to 110° C. in the microwave reactor for 15 minutes. Then the solvent is eliminated in vacuo and the residue is chromatographed on silica gel (toluene/ethyl acetate=96:4). Yield: 0.68 g (10% of theory); C₉H₁₂N₂OS (196.27); mass spectrum: (M+H)⁺=197.

Preparation of the end products:

General method of preparation for Examples 1 to 8

1 mmol of the corresponding norbornane-, norbornene-, or bicyclo[2.2.2]octane-carboxylic acid is dissolved in 5 mL of dichloromethane, combined with 0.3 mL of oxalyl chloride, and stirred for 1 hour at ambient temperature. Then the mixture is evaporated down to the residue. The corresponding acid chloride is added dropwise to a solution of 0.3 g (1 mmol) of 2-amino-3-cyano-4-trifluoromethyl-5-(2,5-dimethylphenyl)thiophene (Example I) and 0.24 mL (3 mmol) of pyridine in 5 mL of dichloromethane cooled to 5° C. The reaction solution is stirred for 12 hours at ambient temperature, diluted with dichloromethane, and washed twice with 1 N hydrochloric acid. The organic phase is dried over magnesium sulfate and evaporated to dryness. The residue is chromatographed on silica gel (cyclohexane/ethyl acetate=9:1).

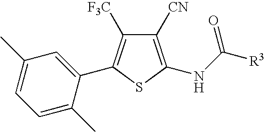

| | | melting point | |
|---|---|---|---|
| Example | R³ | [°C.] | Name |
| 1 | 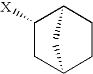 | 242 | 2-[(1S,2S,4R)-bicyclo[2.2.1]hept-2- ylcarbonylamino]-3-cyano-4-trifluoromethyl- 5-(2,5-dimethylphenyl)thiophene |
| 2 | 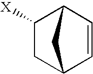 | 209-211 | 2-[(1S,2S,4S)-bicyclo[2.2.1]hept-5-en-2- ylcarbonylamino]-3-cyano-4-trifluoromethyl- 5-(2,5-dimethylphenyl)thiophene |
| 3 | 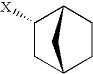 | 230 | 2-[(1R,2S,4S)-bicyclo[2.2.1]hept-2- ylcarbonylamino]-3-cyano-4-trifluoromethyl- 5-(2,5-dimethylphenyl)thiophene |
| 4 | 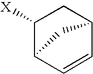 | 226-227 | 2-[(1S,2R,4S)-bicyclo[2.2.1]hept-5-en-2- ylcarbonylamino]-3-cyano-4-trifluoromethyl- 5-(2,5-dimethylphenyl)thiophene |
| 5 | 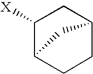 | 239-240 | 2-[(1R,2R,4S)-bicyclo[2.2.1]hept-2- ylcarbonylamino]-3-cyano-4-trifluoromethyl- 5-(2,5-dimethylphenyl)thiophene |
| 6 | 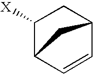 | 213-214 | 2-[(1R,2R,4R)-bicyclo[2.2.1]hept-5-en-2- ylcarbonylamino]-3-cyano-4-trifluoromethyl- 5-(2,5-dimethylphenyl)thiophene |
| 7 | 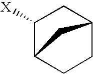 | 231-233 | 2-[(1S,2R,4R)-bicyclo[2.2.1]hept-2- ylcarbonylamino]-3-cyano-4-trifluoromethyl- 5-(2,5-dimethylphenyl)thiophene |
| 8 | 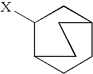 | 242-244 | 2-[bicyclo[2.2.2]oct-2-ylcarbonylamino]-4- chloro-3-cyano-4-trifluoromethyl-5-(2,5- dimethylphenyl)thiophene |

### EXAMPLE 9

### 2-[(1S,2R,4R)-bicyclo[2.2.1]hept-2-ylcarbonylamino]-3-cyano-5-(2,5-dimethyl-phenyl)thiophene

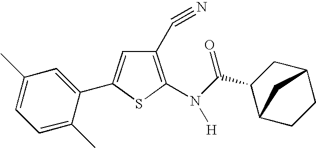 Prepared analogously to Examples 1 to 8 from (+)-(1S,2R,4R)-bicyclo[2.2.1]heptane-2-carboxylic acid and 2-amino-3-cyano-5-(2,5-dimethylphenyl)thiophene (Example I).
Yield: 35% of theory; C₂₁H₂₂N₂OS (350.49); melting point: 168° C.-169° C.; R_{f} value: 0.59 (silica gel; toluene/ethyl acetate=4:1).

### EXAMPLE 10

### 2-[(1S,2R,4R)-bicyclo[2.2.1]hept-2-ylcarbonylamino]-4-chloro-3-cyano-5-(2,5-dimethylphenyl)thiophene

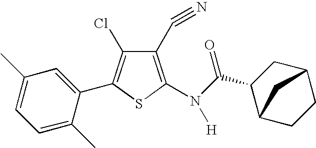 0.5 g (1.4 mmol) of 2-[(1S,2R,4R)-bicyclo[2.2.1]hept-2-ylcarbonylamino]-3-cyano-5-(2,5-dimethylphenyl)thiophene (Example 9) is dissolved in 30 mL of glacial acetic acid at 70° C. and 0.22 g (1.6 mmol) of N-chlorosuccinimide are added. The reaction solution is stirred for 2 hours at 70° C. After cooling, the mixture is made up to 100 mL with water and extracted three times with ethyl acetate. The organic phases are washed twice with 10% sodium carbonate solution and twice with water and then evaporated down to the residue. The residue is chromatographed on silica gel (cyclohexane/ethyl acetate=9:1) and the purified product crystallized from methanol. Yield: 0.1 g (18% of theory); C₂₁H₂₁ClN₂OS (384.92); melting point: 117° C.-119° C.; mass spectrum: (M-H)-=385, 383; R_{f} value: 0.69 (silica gel; toluene/ethyl acetate=4:1).

### EXAMPLE 11

### 2-[(2R)-bicyclo[2.2.2]oct-2-ylcarbonylamino]-4-chloro-3-cyano-5-(2,5-dimethylphenyl)thiophene

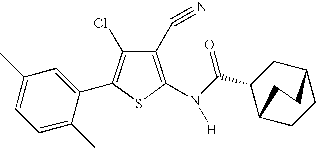 Prepared analogously to Example 10 from 2-[(R)-bicyclo[2.2.2]oct-2-ylcarbonylamino]-3-cyano-5-(2,5-dimethylphenyl)thiophene. Yield: 36% of theory; C₂₂H₂₃ClN₂OS (398.95); melting point: 208° C.-210° C.; mass spectrum: (M+H)⁺=401, 399.

### EXAMPLE 12

### 4-chloro-3-cyano-2-(cyclohexylcarbonylamino)-5-phenylthiophene

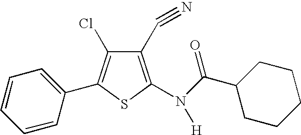

### a. 3-cyano-2-(cyclohexylcarbonylamino)-5-phenylthiophene

Prepared analogously to Examples 1 to 8 from 2-amino-3-cyano-5-phenylthiophene (Example VII) and cyclohexanecarboxylic acid chloride in pyridine. Yield: 81% of theory; C₁₈H₁₈N₂OS (310.41); melting point: 208° C.-210° C.

### b. 4-chloro-3-cyano-2-(cyclohexylcarbonylamino)-5-phenylthiophene

Prepared analogously to Example 10 from 3-cyano-2-(cyclohexylcarbonylamino)-5-phenylthiophene. Yield: 29% of theory; C₁₈H₁₇ClN₂OS (344.86); melting point: 175° C.-180° C.; mass spectrum: (M-H)-=345, 343.

### EXAMPLE 13

### 4-bromo-3-cyano-2-(cyclohexylcarbonylamino)-5-phenylthiophene

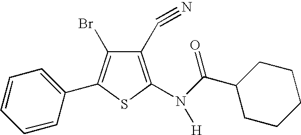 1.1 g (4 mmol) of 3-cyano-2-(cyclohexylcarbonylamino)-5-phenylthiophene (Example 12a) is suspended in 5 mL of glacial acetic acid at 50° C. and within 10 minutes combined with 0.62 g (4 mmol) of N-bromosuccinimide. After another 2 hours' stirring at this temperature and subsequent cooling, the crystals are suction filtered and washed with glacial acetic acid. Yield: 300 mg (22% of theory); C₁₈H₁₇BrN₂OS (389.31); melting point: 167° C.-168° C.; mass spectrum: (M+H)⁺=391, 389.

### EXAMPLE 14

### 3,4-dicyano-2-(cyclohexylcarbonylamino)-5-phenylthiophene

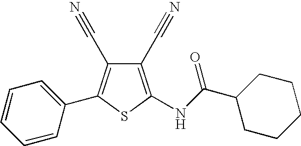 0.75 g (1.9 mmol) of 4-bromo-3-cyano-2-(cyclohexylcarbonylamino)-5-phenylthiophene (Example 13) and 0.26 g (2.9 mmol) of copper (I) cyanide are refluxed for 12 hours in 5 mL of anhydrous DMF. After cooling, the reaction mixture is evaporated down and the residue is chromatographed on silica gel (cyclohexane/ethyl acetate=95:5). The purified product is dissolved in dichloromethane, some methanol is added, and the dichloromethane is eliminated by distillation. The precipitate formed is suction filtered. Yield: 0.29 g (45% of theory); C₁₉H₁₇N₃OS (335.42); melting point: 208° C.-212° C.; mass spectrum: (M+H)⁺=336; R_{f} value: 0.51 (silica gel; toluene/ethyl acetate=4:1).

### EXAMPLE 15

### 3-cyano-2-(cyclohexylcarbonylamino)-4-nitro-5-phenylthiophene

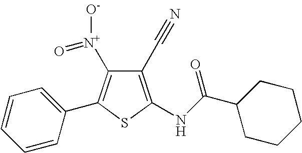 1 g (3 mmol) of 3-cyano-2-(cyclohexylcarbonylamino)-5-phenylthiophene (Example 12a) is placed in 80 mL of acetic anhydride. At 5° C., 4 mL of 60% nitric acid is slowly added dropwise. After 10 minutes stirring at this temperature, the solution is heated to ambient temperature and stirred for a further 2 hours. The reaction mixture is added to 10 mL of ice and stirred. Then the phases are separated. The aqueous phase is extracted with dichloromethane. The combined organic phases are dried over magnesium sulfate and evaporated down. The residue is combined with methanol and dichloromethane, the dichloromethane is distilled off and the precipitate formed is suction filtered. Yield: 0.1 g (9% of theory); C₁₈H₁₇N₃O₃S (355.41); melting point: 213° C.-214° C.; mass spectrum: (M−H)⁻=354; R_{f} value: 0.63 (silica gel; toluene/ethyl acetate=4:1).

### EXAMPLE 16

### 3-cyano-2-(cyclohexylcarbonylamino)-4-methoxy-5-propylthiophene

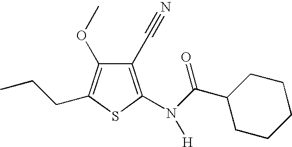 0.68 g (3.5 mmol) of 2-amino-3-cyano-4-methoxy-5-propylthiophene (Example VIII) and 0.84 mL (10.4 mmol) of pyridine are dissolved in 25 mL of dichloromethane. Within 10 minutes 0.48 mL of cyclohexanecarboxylic acid chloride in 5 mL of dichloromethane are added dropwise at 10° C. The solution is stirred for 12 hours at ambient temperature and extracted with dilute hydrochloric acid and with water, dried over magnesium sulfate, and evaporated down. The residue is chromatographed on silica gel (toluene/ethyl acetate=95:5). The isolated product is recrystallized from diisopropylether. Yield: 0.32 g (30% of theory); C₁₆H₂₂N₂O₂S (306.42); melting point: 146° C.-147° C.; mass spectrum: (M+H)⁺=307.

### EXAMPLE 17

### 2-[(1 S,2R,4R)-bicyclo[2.2.1]hept-2-ylcarbonylamino]4-bromo-3-cyano-5-(2,5-dimethylphenyl)thiophene

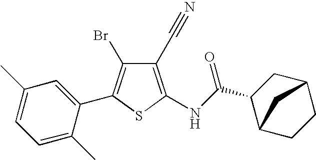 Prepared analogously to Examples 1 to 8 from 2-amino-4-bromo-3-cyano-5-(2,5-dimethyl-phenyl)thiophene (Example IV) and (1S,2R,4R)-bicyclo[2.2.1]heptane-2-carboxylic acid chloride. Yield: 63% of theory; C₂₁H₂₁BrN₂OS (429.37); mass spectrum: (M+H)⁺=431, 429; R_{f} value: 0.72 (silica gel; petroleum ether/ethyl acetate=4:1).

### EXAMPLE 18

### 2-[(1S,2R,4R)-bicyclo[2.2.1]hept-2-ylcarbonylamino]-4-bromo-3-cyano-5-{3-[methyl-(4-methylphenyl)amino]propyl}thiophene hydrochloride

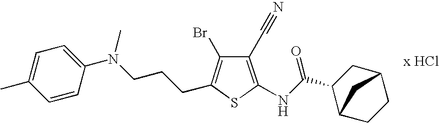

### a. 2-[(1S,2R,4R)-bicyclo[2.2.1]hept-2-ylcarbonylamino]-5-(3-chloropropyl)-3-cyanothiophene

Prepared analogously to Examples 1 to 8 from 2-amino-5-(3-chloropropyl)-3-cyanothiophene (Example V) and (1S,2R,4R)-bicyclo[2.2.1]heptane-2-carboxylic acid chloride. Yield: 48% of theory; C₁₆H₁₉ClN₂OS (322.85); mass spectrum: (M+H)⁺=325, 323; R_{f} value: 0.58 (silica gel; petroleum ether/ethyl acetate=3:1).

### b. 2-[(1S,2R,4R)-bicyclo[2.2.1 hept-2-ylcarbonylamino]-4-bromo-5-(3-chloropropyl)-3-cyanothiophene

A solution of 100 mg (0.31 mmol) of 2-[(1S,2R,4R)-bicyclo[2.2.1]hept-2-ylcarbonyl-amino]-5-(3-chloropropyl)-3-cyanothiophene in 0.4 mL of dichloromethane is combined with 82 mg (0.62 mmol) of anhydrous aluminum chloride. Then 18 μL (0.34 mmol) of bromine is added. The mixture is refluxed for 3 hours. Then another 50 mg (0.38 mmol) of aluminum chloride and 10 μL (0.19 mmol) of bromine are added. The mixture is stirred for 16 hours at ambient temperature and refluxed again for 2 hours. Then the reaction solution is poured onto 5% sodium bisulfite solution. The mixture is extracted with ethyl acetate, and the organic phase is washed with saturated sodium chloride solution and dried over magnesium sulfate. The crude product obtained (110 mg) after removal of the solvent is further reacted immediately.

### c. 2-[(1S,2R,4R)-bicyclo[2.2.1]hept-2-ylcarbonylamino]-4-bromo-3-cyano-5-{3-[methyl-(4-methylphenyl)amino]propyl}thiophene hydrochloride

110 mg (0.271 mmol) of 2-[(1S,2R,4R)-bicyclo[2.2.1]hept-2-ylcarbonylamino]4-bromo-5-(3-chloropropyl)-3-cyanothiophene is combined with 0.6 mL (4.6 mmol) of N-methyl-p-toluidine and heated to 160° C. in the microwave reactor for 45 minutes. Then 2.5 mL of 2 N hydrochloric acid and 5 mL of water are added. The reaction solution is extracted with ethyl acetate. The organic phase is washed with water, 2 N soda solution, water, and saturated sodium chloride solution and dried over magnesium sulfate. The residue obtained after removal of the solvent is purified by preparative HPLC (Agilent). Yield: 13 mg (9% of theory over 2 steps); C₂₄H₂₈BrN₃OS×HCl (522.93); mass spectrum: (M+H)⁺=488, 486.

### EXAMPLE 19

### 2-[(1S,2R,4R)-bicyclo[2.2.1]hept-2-ylcarbonylamino]-3,4-dicyano-5-{3-[methyl-(4-methylphenyl)amino]propyl}thiophene hydrochloride

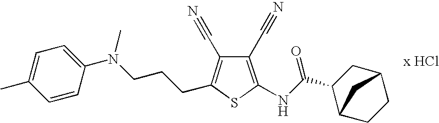 Prepared analogously to Example 14 from 2-[(1S,2R,4R)-bicyclo[2.2.1]hept-2-ylcarbonyl-amino]-4-bromo-3-cyano-5-{3-[methyl-(4-methylphenyl)amino]propyl}thiophene hydrochloride (Example 18) and copper (I) cyanide in DMF. Yield: 16% of theory; C₂₅H₂₈N₄OS×HCl (469.04); mass spectrum: (M+H)⁺=433.

### EXAMPLE 20

### 2-[(1S,2R,4R)-bicyclo[2.2.1]hept-2-ylcarbonylamino]-5-(3-chloropropyl)-3-cyano-4-methylthiophene

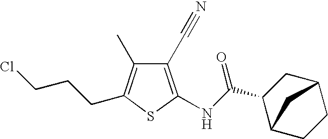 Prepared analogously to Examples 1 to 8 from 2-amino-5-(3-chloropropyl)-3-cyano-5-methylthiophene (Example VI) and (1S,2R,4R)-bicyclo[2.2.1]heptane-2-carboxylic acid chloride. Yield: 43% of theory; C₁₇H₂₁ClN₂OS (336.88); mass spectrum: (M+H)⁺=339, 337; R_{f} value: 0.46 (silica gel; petroleum ether/ethyl acetate=4:1).

### EXAMPLE 21

### 2-[(1S,2R,4R)-bicyclo[2.2.1]hept-2-ylcarbonylamino]-3-cyano-4-methyl-5-{3-[methyl-(4-methylphenyl)amino]propyl}thiophene hydrochloride

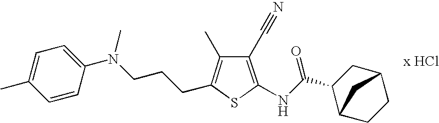 Prepared analogously to Example 18c from 2-[(1S,2R,4R)-bicyclo[2.2.1]hept-2-ylcarbonyl-amino]-5-(3-chloropropyl)-3-cyano-4-methylthiophene (Example 20) and N-methyl-p-toluidine. Yield: 48% of theory; C₂₅H₃₁N₃OS×HCl (458.06); melting point: 116° C.-129° C. (decomposition); mass spectrum: (M+H)⁺=422.

### EXAMPLE 22

### 2-[(1S,2R,4R)-bicyclo[2.2.1]hept-2-ylcarbonylamino]-3-cyano-4-methyl-5-{3-[methyl-(2-pyridyl)amino]propyl}thiophene dihydrochloride

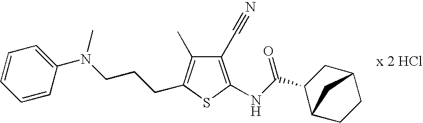 Prepared analogously to Example 18c from 2-[(1S,2R,4R)-bicyclo[2.2.1]hept-2-ylcarbonyl-amino]-5-(3-chloropropyl)-3-cyano-4-methylthiophene (Example 20) and 2-(methylamino)pyridine. Yield: 12% of theory; C₂₃H₂₈N₄OS×2HCl (481.48); mass spectrum: (M+H)⁺=409.

### EXAMPLE 23

### 2-[bicyclo]2.2.2[oct-2-ylcarbonylamino]-5-(3-chloropropyl)-3-cyano-4-methylthiophene

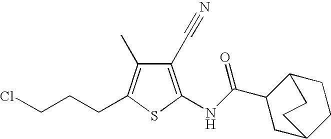 Prepared analogously to Examples 1 to 8 from 2-amino-5-(3-chloropropyl)-3-cyano-4-methylthiophene (Example VI) and bicyclo[2.2.2]octane-2-carboxylic acid chloride.
Yield: 82% of theory; C₁₈H₂₃ClN₂OS (350.91); mass spectrum: (M+H)⁺=353, 351; R_{f} value: 0.56 (silica gel; petroleum ether/ethyl acetate=4:1).

### EXAMPLE 24

### 2-(bicyclo[2.2.2]oct-2-ylcarbonylamino)-3-cyano-4-methyl-5-{3-[methyl-(4-methylphenyl)amino]propyl}thiophene hydrochloride

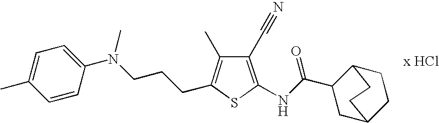 Prepared analogously to Example 18c from 2-(bicyclo[2.2.2]oct-2-ylcarbonylamino)-5-(3-chloropropyl)-3-cyano-4-methylthiophene (Example 23) and N-methyl-p-toluidine. Yield: 47% of theory; C₂₆H₃₃N₃OS×HCl (472.09); melting point: 133° C.-140° C. (strong sintering from 125° C.); mass spectrum: (M+H)⁺=436.

### EXAMPLE 25

### 2-(bicyclo[2.2.2]oct-2-ylcarbonylamino]-3-cyano-4-methyl-5-{3-[methyl-(2-pyridyl)amino]propyl}thiophene dihydrochloride

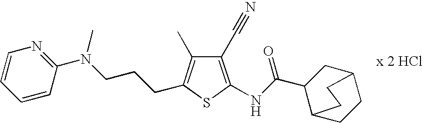

### a. 2-(bicyclo[2.2.2]oct-2-ylcarbonylamino]-3-cyano-4-methyl-5-[3-(N-methylallylamino)-propyl]thiophene

Prepared analogously to Example 18c from 2-(bicyclo[2.2.2]oct-2-ylcarbonylamino)-5-(3-chloropropyl)-3-cyano-4-methylthiophene (Example 23) and N-methylallylamine. Yield: 98% of theory; C₂₂H₃₁N₃OS (385.57); mass spectrum: (M+H)⁺=386.

### b. 2-(bicyclo[2.2.2]oct-2-ylcarbonylamino]-3-cyano-4-methyl-5-(3-methylaminopropyl)thiophene

696 mg (4.46 mmol) of 1,3-dimethylbarbituric acid and 17 mg (0.015 mmol) of tetrakis-(triphenylphosphine)palladium(0) are added under a nitrogen atmosphere to a solution of 573 mg (1.49 mmol) of 2-(bicyclo[2.2.2]oct-2-ylcarbonylamino]-3-cyano-4-methyl-5-[3-(N-methylallylamino)propyl]thiophene in 3.5 mL of dichloromethane. The reaction mixture is heated to 35° C.-40° C. for 16 hours. Then the solvent is eliminated in vacuo and the residue is taken up in saturated sodium carbonate solution. The mixture is extracted with ether/dichloromethane, the organic phase is washed with saturated saline solution, dried over magnesium sulfate, and the solvent is eliminated in vacuo. The crude product thus obtained (560 mg) is further reacted immediately. C₁₉H₂₇N₃OS (345.50); mass spectrum: (M+H)⁺=346.

### c. 2-(bicyclo[2.2.2]oct-2-ylcarbonylamino]-3-cyano-4-methyl-5-{3-[methyl-(2-pyridyl)amino]propyl}thiophene dihydrochloride

200 mg (0.579 mmol) of 2-(bicyclo[2.2.2]oct-2-ylcarbonylamino]-3-cyano-4-methyl-5-(3-methylaminopropyl)thiophene is dissolved in 1.5 mL of DMF under a nitrogen atmosphere and combined with 0.17 mL (1.78 mmol) of 2-bromopyridine and 160 mg (1.16 mmol) of potassium carbonate. The mixture is heated to 100° C. in the microwave for 60 minutes. After the addition of another 0.1 mL (1.05 mmol) of 2-bromopyridine, the mixture is again heated to 130° C. for 6 hours in the microwave. After filtration to remove insoluble matter the solvent is eliminated in vacuo and the residue is purified by preparative HPLC (Agilent, RP phase). Yield: 59 mg (21% of theory); C₂₄H₃₀N₄OS×2 HCl (495.51); melting point: >80° C. (decomposition); mass spectrum: (M+H)⁺=423.

### EXAMPLE 26

### Tablets

per tablet:

| | |
|---|---|
| active substance of formula (I) | 100 mg |
| lactose | 140 mg |
| corn starch | 240 mg |
| polyvinylpyrrolidone | 15 mg |
| magnesium stearate | 5 mg |
| TOTAL | 500 mg |

The finely ground active substance, lactose and some of the corn starch are mixed together. The mixture is screened, then moistened with a solution of polyvinylpyrrolidone in water, kneaded, wet-granulated, and dried. The granules, the remaining corn starch and the magnesium stearate are screened and mixed together. The mixture is compressed to produce tablets of suitable shape and size.

### EXAMPLE 27

### Tablets

per tablet:

| | |
|---|---|
| active substance | 80 mg |
| corn starch | 190 mg |
| lactose | 55 mg |
| microcrystalline cellulose | 35 mg |
| polyvinylpyrrolidone | 15 mg |
| sodium-carboxymethyl starch | 23 mg |
| magnesium stearate | 2 mg |
| TOTAL | 400 mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodium-carboxymethyl starch and the magnesium stearate are added and mixed in and the mixture is compressed to form tablets of a suitable size.

### EXAMPLE 28

### Coated Tablets

per coated tablet:

| | |
|---|---|
| active substance | 5 mg |
| corn starch | 41.5 mg |
| lactose | 30 mg |
| polyvinylpyrrolidone | 3 mg |
| magnesium stearate | 0.5 mg |
| TOTAL | 80 mg |

The active substance, corn starch, lactose and polyvinylpyrrolidone are thoroughly mixed and moistened with water. The moist mass is pushed through a screen with a 1 mm mesh size, dried at about 45° C. and the granules are then passed through the same screen. After the magnesium stearate has been mixed in, convex tablet cores with a diameter of 6 mm are compressed in a tablet-making machine. The tablet cores thus produced are coated in a known manner with a covering consisting essentially of sugar and talc. The finished coated tablets are polished with wax.

### EXAMPLE 29

### Capsules

per capsule:

| | |
|---|---|
| active substance | 50 mg |
| corn starch | 268.5 mg |
| magnesium stearate | 1.5 mg |
| TOTAL | 320 mg |

The substance and corn starch are mixed and moistened with water. The moist mass is screened and dried. The dry granules are screened and mixed with magnesium stearate. The finished mixture is packed into size 1 hard gelatine capsules.

### EXAMPLE 30

### Ampoule Solution

| | | |
|---|---|---|
| active substance | 50 | mg |
| sodium chloride | 50 | mg |
| water for inj. | 5 | mL |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilized and sealed by fusion. The ampoules contain 5 mg, 25 mg, and 50 mg of active substance.

### EXAMPLE 31

### Suppositories

| | |
|---|---|
| active substance | 50 mg |
| solid fat | 1650 mg |
| TOTAL | 1700 mg |

The hard fat is melted. At 40° C., the ground active substance is homogeneously dispersed therein. It is cooled to 38° C. and poured into slightly chilled suppository moulds.

### EXAMPLE 32

### Coated Tablets Containing 75 mg of Active Substance

1 tablet core contains:

| | |
|---|---|
| active substance | 75.0 mg |
| calcium phosphate | 93.0 mg |
| corn starch | 35.5 mg |
| polyvinylpyrrolidone | 10.0 mg |
| hydroxypropylmethylcellulose | 15.0 mg |
| magnesium stearate | 1.5 mg |
| TOTAL | 230.0 mg |

Preparation: The active substance is mixed with calcium phosphate, corn starch, polyvinylpyrrolidone, hydroxypropylmethylcellulose and half the specified amount of magnesium stearate. Blanks approx. 13 mm in diameter are produced in a tablet-making machine and these are then rubbed through a screen with a mesh size of 1.5 mm using a suitable machine and mixed with the rest of the magnesium stearate. This granulate is compressed in a tablet-making machine to form tablets of the desired shape. Weight of core: 230 mg; die: 9 mm, convex. The tablet cores thus produced are coated with a film consisting essentially of hydroxypropylmethylcellulose. The finished film-coated tablets are polished with beeswax. Weight of coated tablet: 245 mg.

### EXAMPLE 33

### Tablets Containing 100 mg of Active Substance

1 tablet contains:

| | |
|---|---|
| active substance | 100.0 mg |
| lactose | 80.0 mg |
| corn starch | 34.0 mg |
| polyvinylpyrrolidone | 4.0 mg |
| magnesium stearate | 2.0 mg |
| TOTAL | 220.0 mg |

Preparation: The active substance, lactose and starch are mixed together and uniformly moistened with an aqueous solution of the polyvinylpyrrolidone. After the moist composition has been screened (2.0 mm mesh size) and dried in a rack-type drier at 50° C., it is screened again (1.5 mm mesh size) and the lubricant is added. The finished mixture is compressed to form tablets. Weight of tablet: 220 mg; diameter: 10 mm, biplanar, facetted on both sides and notched on one side.

### EXAMPLE 34

### Tablets Containing 150 mg of Active Substance

1 tablet contains:

| | |
|---|---|
| active substance | 150.0 mg |
| powdered lactose | 89.0 mg |
| corn starch | 40.0 mg |
| colloidal silica | 10.0 mg |
| polyvinylpyrrolidone | 10.0 mg |
| magnesium stearate | 1.0 mg |
| TOTAL | 300.0 mg |

Preparation: The active substance mixed with lactose, corn starch and silica is moistened with a 20% aqueous polyvinylpyrrolidone solution and passed through a screen with a mesh size of 1.5 mm. The granules, dried at 45° C., are passed through the same screen again and mixed with the specified amount of magnesium stearate. Tablets are pressed from the mixture. Weight of tablet: 300 mg; die: 10 mm, flat.

### EXAMPLE 35

### Hard Gelatine Capsules Containing 150 mg of Active Substance

1 capsule contains:

| | | |
|---|---|---|
| active substance | 150.0 | mg |
| corn starch (dried) | approx. 180.0 | mg |
| lactose (powdered) | approx. 87.0 | mg |
| magnesium stearate | 3.0 | mg |
| TOTAL | approx. 420.0 | mg |

Preparation: The active substance is mixed with the excipients, passed through a screen with a mesh size of 0.75 mm and homogeneously mixed using a suitable apparatus. The finished mixture is packed into size 1 hard gelatine capsules. Capsule filling: approx. 320 mg; Capsule shell: size 1 hard gelatine capsule.

### EXAMPLE 36

### Suppositories Containing 150 mg of Active Substance

1 suppository contains:

| | |
|---|---|
| active substance | 150.0 mg |
| polyethyleneglycol 1500 | 550.0 mg |
| polyethyleneglycol 6000 | 460.0 mg |
| polyoxyethylene sorbitan monostearate | 840.0 mg |
| TOTAL | 2,000.0 mg |

Preparation: After the suppository mass has been melted, the active substance is homogeneously distributed therein and the melt is poured into chilled moulds.

### EXAMPLE 37

### Suspension Containing 50 mg of Active Substance

100 mL of suspension contains:

| | | |
|---|---|---|
| active substance | 1.00 | g |
| carboxymethylcellulose-Na-salt | 0.10 | g |
| methyl p-hydroxybenzoate | 0.05 | g |
| propyl p-hydroxybenzoate | 0.01 | g |
| glucose | 10.00 | g |
| glycerol | 5.00 | g |
| 70% sorbitol solution | 20.00 | g |
| flavoring | 0.30 | g |
| dist. water | to 100 | mL |

Preparation: The distilled water is heated to 70° C. The methyl and propyl p-hydroxybenzoates together with the glycerol and sodium salt of carboxymethylcellulose are dissolved therein with stirring. The solution is cooled to ambient temperature and the active substance is added and homogeneously dispersed therein with stirring. After the sugar, the sorbitol solution, and the flavoring have been added and dissolved, the suspension is evacuated with stirring to eliminate air. 5 mL of suspension contains 50 mg of active substance.

### EXAMPLE 38

### Ampoules Containing 10 mg Active Substance

Composition:

| | |
|---|---|
| active substance | 10.0 mg |
| 0.01 N hydrochloric acid | q.s. |
| double-distilled water | to 2.0 mL |

Preparation: The active substance is dissolved in the necessary amount of 0.01 N HCl, made isotonic with common salt, filtered sterile and transferred into 2 mL ampoules.

### EXAMPLE 39

### Ampoules Containing 50 mg of Active Substance

Composition:

| | |
|---|---|
| active substance | 50.0 mg |
| 0.01 N hydrochloric acid | q.s. |
| double-distilled water | to 10.0 mL |

Preparation: The active substance is dissolved in the necessary amount of 0.01 N HCl, made isotonic with common salt, filtered sterile, and transferred into 10 mL ampoules.

We claim:

1. A compound of formula (I) 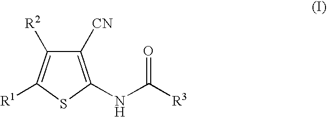
wherein:
R¹ is a C₁₋₄-alkyl group optionally terminally substituted by a fluorine, chlorine, bromine, or iodine atom or by an amino group, wherein the hydrogen atoms of the amino group are optionally independently substituted by a C₁₋₃-alkyl group or by a phenyl or pyridyl group optionally substituted by a C₁₋₃-alkyl group, or
a phenyl or pyridyl group each optionally substituted by one to three C₁₋₃-alkyl groups;
R² is a hydrogen, fluorine, chlorine, bromine, or iodine atom, or a cyano, nitro, C₁₋₃-alkyl, trifluoromethyl, or C₁₋₄-alkyloxy group; and
R³ is a bicyclo[2.2.1]hept-2-enyl group or a cyclohexyl group wherein the carbon atom in position 2 is optionally bridged to the carbon atom in position 5 by a —CH₂—, —CH₂—CH₂—, or —CH═CH group,
or a tautomer, enantiomer, or salt thereof.

2. The compound of formula (I) according to claim 1, wherein R² is a fluorine, chlorine, bromine, or iodine atom or a cyano, nitro, C₁₋₃-alkyl, trifluoromethyl or C₁₋₄-alkyloxy group, or a tautomer, enantiomer, or salt thereof.

3. The compound of formula (I) according to claim 2, wherein: R² is a fluorine, chlorine, bromine, or iodine atom or a cyano, nitro, or C₁₋₄-alkyloxy group, or a tautomer, enantiomer, or salt thereof.

4. The compound of formula (I) according to claim 1, wherein R³ is a cyclohexyl group wherein the carbon atom in position 2 is optionally bridged to the carbon atom in position 5 by a —CH₂— or —CH₂—CH₂ group, or a tautomer, enantiomer, or salt thereof.

5. The compound of formula (I) according to claim 1, wherein
R¹ is a C₁₋₄-alkyl group optionally terminally substituted by a fluorine, chlorine, bromine, or iodine atom or by an amino group, wherein the hydrogen atoms of the amino group are optionally independently substituted by a C₁₋₃-alkyl group or by a phenyl or pyridyl group optionally substituted by a C₁₋₃-alkyl group, or
a phenyl or pyridyl group each optionally substituted by one or two C₁₋₃-alkyl groups; and
R³ is a cyclohexyl group wherein the carbon atom in position 2 is optionally bridged to the carbon atom in position 5 by a —CH₂— or —CH₂—CH₂ group,
or a tautomer, enantiomer, or salt thereof.

6. The compound of formula (I) according to claim 1, wherein:
R¹ is a C₂₋₄-alkyl group terminally substituted by a N-phenyl-N-methylamino group, or a phenyl group optionally substituted by one or two methyl groups;
R² is a chlorine or bromine atom or a cyano or nitro group; and
R³ is a cyclohexyl group wherein the carbon atom in position 2 is optionally bridged to the carbon atom in position 5 by a —CH₂— or —CH₂—CH₂— group,
or a tautomer, enantiomer, or salt thereof.

7. A compound of selected from:
(a) 2-[(1S,2R,4R)-bicyclo[2.2.1]hept-2-ylcarbonylamino]-4-chloro-3-cyano-5-(2,5-dimethylphenyl)thiophene,
(b) 2-[(2R)-bicyclo[2.2.2]oct-2-ylcarbonylamino]-4-chloro-3-cyano-5-(2,5-dimethyl-phenyl)thiophene,
(c) 4-chloro-3-cyano-2-(cyclohexylcarbonylamino)-5-phenylthiophene,
(d) 4-bromo-3-cyano-2-(cyclohexylcarbonylamino)-5-phenylthiophene,
(e) 3,4-dicyano-2-(cyclohexylcarbonylamino)-5-phenylthiophene,
(f) 3-cyano-2-(cyclohexylcarbonylamino)-4-nitro-5-phenylthiophene,
(g) 2-[(1S,2R,4R)-bicyclo[2.2.1]hept-2-ylcarbonylamino]-4-bromo-3-cyano-5-(2,5-dimethylphenyl)thiophene,
(h) 2-[(1S,2R,4R)-bicyclo[2.2.1]hept-2-ylcarbonylamino]-4-bromo-3-cyano-5-{3-[methyl-(4-methylphenyl)amino]propyl}thiophene and
(i) 2-[(1S,2R,4R)-bicyclo[2.2.1]hept-2-ylcarbonylamino]-3,4-dicyano-5-{3-[methyl-(4-methylphenyl)amino]propyl}thiophene
or a tautomer, enantiomer, or salt thereof.

8. A physiologically acceptable salt of the compound according to claim 1.

9. A physiologically acceptable salt of the compound according to claim 2.

10. A physiologically acceptable salt of the compound according to claim 3.

11. A physiologically acceptable salt of the compound according to claim 4.

12. A physiologically acceptable salt of the compound according to claim 5.

13. A physiologically acceptable salt of the compound according to claim 6.

14. A physiologically acceptable salt of the compound according to claim 7.

15. A pharmaceutical composition comprising the compound according to claim 1 and one or more inert carriers and/or diluents.

16. A pharmaceutical composition comprising the physiologically acceptable salt according to claim 8 and one or more inert carriers and/or diluents.
